(19) United States
(12) Patent Application Publication
(10) Pub. No.: US 2012/0041211 A1
(43) Pub. Date: Feb. 16, 2012

(54) NOVEL PROCESS FOR PREPARING CARBOXY-CONTAINING PYRAZOLEAMIDO COMPOUNDS 597

(76) Inventors: Sythana Suresh Kumar, Bangalore (IN); Vinod Ch Kumar, Bangalore (IN); Paramashivappa Rangappa, Bangalore (IN)

(21) Appl. No.: 13/146,762

(22) PCT Filed: Jan. 29, 2010
(86) PCT No.: PCT/SE2010/050093

§ 371 Date: Oct. 11, 2011

(30) Foreign Application Priority Data
Jan. 30, 2009 (PK) 083/2009
Mar. 3, 2009 (GB) 0903631.0

(51) Int. Cl. C07D 231/14; C07C 231/02

(52) U.S. Cl. 548/374.1; 564/136

(57) ABSTRACT

A process for preparing pharmaceutically acceptable compounds of formula (I)

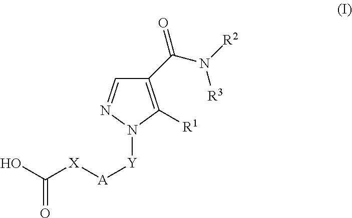

wherein R¹, R², R³, X, A and Y are as defined in the specification is described and claimed, together with processes for preparing some key intermediates and products obtained thereby.

The present invention relates to processes for the preparation of a range of pharmaceutical compounds and intermediates used in the preparation.

WO2008/099145 discloses a range of chemical compounds, or pharmaceutically-acceptable salts thereof that possess human 11-β-hydroxysteroid dehydrogenase type 1 enzyme (11βHSD1) inhibitory activity and accordingly have value in the treatment of disease states including metabolic syndrome and are useful in methods of treatment of a warm-blooded animal, such as man. The invention also relates to processes for the manufacture of said compounds, to pharmaceutical compositions containing them and to their use in the manufacture of medicaments to inhibit 11βHSD1 in a warm-blooded animal, such as man.

In particular, the compounds of formula (1):

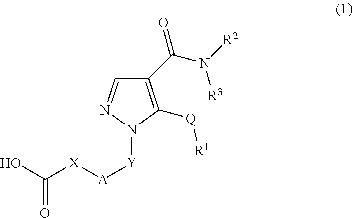

wherein:
Q, R¹, R², R³, X, Y and A are as defined in WO2008/099145,
or a pharmaceutically-acceptable salt thereof, are prepared by for example, by hydrolysis of an ester of formula (2):

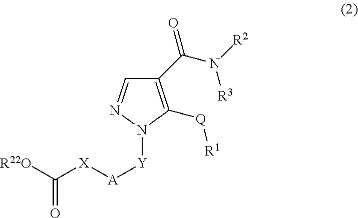

wherein R²² is an alkyl or aryl group and R¹, R², R³, Q, A and X are as defined in relation to formula (I).

It has been found however that there are some problems associated with the preparation of intermediates of formula (2) when they are produced on a large scale. In particular, the synthesis of esters of formula (2) may be lengthy in that esters of starting materials such as compounds of formula (8)

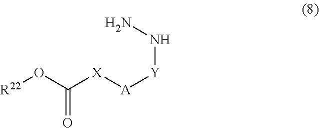

where X, A and R²² are as defined above, may be required to be produced specifically.

Furthermore, cyclisation reactions for example between compounds of formula (12)

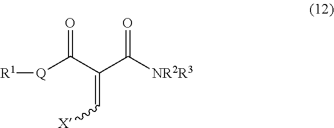

where R², R³, R¹, X′ and Q are as defined in WO2008/099145 and (8) to yield compounds of formula (2) as recommended in WO2008/099145 may require large volumes, for example up to 200 relative volumes of solvent, such as methanol. Such large volume reactions are inefficient and wasteful of solvent.

According to the present invention there is provided a process for preparing a compound of formula (I)

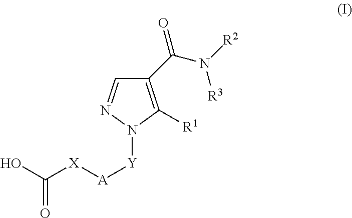

wherein:
R¹ is C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, C₃₋₇cycloalkylC₁₋₃alkyl, C₃₋₇cycloalkylC₂₋₃alkenyl or C₃₋₇cycloalkylC₂₋₃alkynyl, [each of which is optionally substituted by 1, 2 or 3 substituents independently selected from C₁₋₃alkyl, hydroxy, halo, oxo, cyano, trifluoromethyl, C₁₋₃alkoxy, C₁₋₃alkylS(O)ₙ— (wherein n is 0, 1, 2 or 3), R⁵CON(R^{5′})—, (R^{5′})(R^{5″})NC(O)—, R^{5′}C(O)O—, R^{5′}OC(O)—, (R^{5′})(R^{5″})NC(O)N(R^{5′″})—, R⁵SO₂N(R^{5″})—, and (R^{5′})(R^{5″})NSO₂— (wherein R⁵ is C₁₋₃alkyl optionally substituted by 1, 2 or 3 substituents selected from hydroxyl, halo or cyano; and
R^{5′} and R^{5″} are independently selected from hydrogen and C₁₋₃alkyl optionally substituted by 1, 2 or 3 substituents independently selected from hydroxyl, halo, C₁₋₃alkoxy, carboxy and cyano or R^{5′} and R^{5″} together with the nitrogen atom to which they are attached form a 4-7 membered saturated ring)];
R² is selected from heterocyclyl, C₃₋₇cycloalkyl(CH₂)ₘ—, and C₆₋₁₂polycycloalkyl(CH₂)ₘ— (wherein m is 0, 1 or 2 and the rings are optionally substituted by 1, 2 or 3 substituents independently selected from R⁶);
R³ is selected from hydrogen, C₁₋₄alkyl C₃₋₅cycloalkyl and C₃₋₅cycloalkylmethyl (each of which is optionally substituted by 1, 2 or 3 fluoro atoms);
R² and R³ together with the nitrogen atom to which they are attached form a saturated mono, bicyclic or bridged ring system optionally containing 1 or 2 additional ring heteroatoms selected from nitrogen, oxygen and sulphur and which is optionally fused to a saturated, partially saturated or unsaturated monocyclic ring wherein the resulting ring system is optionally substituted by 1, 2, or 3 substituents independently selected from R⁷;
R⁶ and R⁷ are independently selected from hydroxyl, halo, oxo, carboxy, cyano, trifluoromethyl, R⁹, R⁹O—, R⁹CO—, R⁹C(O)O—, R⁹CON(R^{9′})—, (R^{9′})(R^{9″})NC(O)—, (R^{9′})(R^{9″})N—, R⁹S(O)ₐ— wherein a is 0 to 2, R^{9′}OC(O)—, (R^{9′})(R^{9″})NSO₂—, R⁹SO₂N(R^{9″})—, (R^{9′})(R^{9″})NC(O)N(R^{9′″})—, phenyl and heteroaryl [wherein the phenyl and heteroaryl groups are optionally fused to a phenyl, heteroaryl or a saturated or partially-saturated 5- or 6-membered ring optionally containing 1, 2 or 3 heteroatoms independently selected from nitrogen, oxygen and sulphur and the resulting ring system is optionally substituted by 1, 2 or 3 substituents independently selected from C₁₋₄alkyl, hydroxyl, cyano, trifluoromethyl, trifluoromethoxy, halo, C₁₋₄alkoxy, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)ᵣ—, C₁₋₄alkylS(O)ᵣC₁₋₄alkyl (wherein r is 0, 1 or 2)];
R⁹ is independently selected from C₁₋₃alkyl optionally substituted by hydroxyl, halo, C₁₋₄alkoxy, carboxy or cyano;
R^{9′}, R^{9″} and R^{9′″} are independently selected from hydrogen and C₁₋₃alkyl optionally substituted by 1, 2, or 3 substituents independently selected from hydroxyl, halo, C₁₋₄alkoxy, carboxy and cyano);
A is a phenyl or heteroaryl ring (the phenyl or heteroaryl ring being optionally substituted on ring carbon atoms by 1, 2 or 3 R¹⁰ groups and on an available ring nitrogen in a heteroaryl group by R¹¹);
R¹⁰ is independently selected from C₁₋₄alkyl, hydroxyl, cyano, trifluoromethyl, trifluoromethoxy, halo, C₁₋₄alkoxy, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)ₛ—, C₁₋₄alkylS(O)ₛC₁₋₄alkyl (wherein s is 0, 1 or 2)];
R¹¹ is independently C₁₋₃alkyl optionally substituted by 1, 2 or 3 fluoro atoms;
X is a direct bond, C₃₋₄cycloalkandiyl, C₃₋₄cycloalkanylidene, —C(R¹²)(R¹³)—, —C(R¹²)(R¹³)C(R¹⁴)(R¹⁵)—, —CH₂O— or —CH₂S(O)ₜ— (wherein t is 0, 1 or 2):
Y is a direct bond, C₃₋₄cycloalkandiyl, C₃₋₄cycloalkanylidene, —C(R¹⁶)(R¹⁷)— or —C(R¹⁸)(R¹⁹)C(R²⁰)(R²¹)—;
wherein R¹², R¹³, R¹⁴, R¹⁵, R¹⁶, R¹⁷, R¹⁸, R¹⁹, R²⁰ and R²¹ are independently selected from hydrogen and methyl; which process comprises reacting a compound of formula (II)

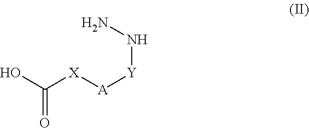

where X and A are as defined in relation to formula (I), with a compound of formula (III)

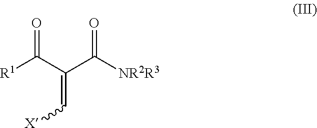

wherein R¹, R² and R³ are as defined above, and X′ represents either dialkylamino (such as dimethylamino) or lower alkoxy (such as methoxy or ethoxy);
and thereafter if necessary or desirable carrying out one or more or the following steps:
i) converting a compound of the formula (1) into another compound of the formula (1);
ii) removing any protecting groups;
iii) resolving enantiomers;
iv) forming a pharmaceutically-acceptable salt thereof;
v) purifying the product.

The process has been found to be efficient in allowing compounds to be prepared directly from acids of formula (II), which may be available commercially. Furthermore, the reaction, which is generally carried out in an organic solvent such as methanol, appears to be require far less solvent than processes where the corresponding esters of formula (II) as shown as compound (8) above are used.

The process is suitably carried out using a suitable solvent such as methanol for example. Typically the reaction is carried out at ambient temperature, although elevated temperatures may be employed, for example the reflux temperature of the solvent. The reaction may be carried out in the presence of an acid such as hydrochloric acid as illustrated in the Examples below.

Examples of conversions of a compound of Formula (I) into another compound of Formula (I), well known to those skilled in the art, include functional group interconversions such as hydrolysis, hydrogenation, hydrogenolysis, oxidation or reduction, and/or further functionalisation by standard reactions such as amide or metal-catalysed coupling, or nucleophilic displacement reactions.

Purification procedures would also be well understood in the art.

Suitably, a purification step in which the product is dissolved in aqueous base such as aqueous sodium hydroxide and insoluble impurities removed by toluene extraction before acidifying the solution to recover the product is used.

Hydrazines of formula (II) are known in the chemical literature or may be prepared using standard conditions known to those skilled in the art.

Compounds of formula (III) may also be prepared by processes known in the art, for example as described in WO2008/099145. They are suitably prepared by reacting a compound of formula (IV)

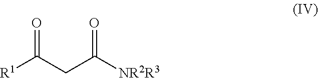

where R¹, R² and R³ are as defined above, with an acetal of formula (V)

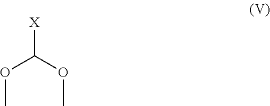

where X′ is as defined above. The reaction is suitably carried out in an organic solvent at temperatures in the range of from 85 to 95° C. Although WO2008/099145 suggests the use of 1,4-dioxane as a solvent, and treatment at high temperatures for example of 100° C. under nitrogen, followed by evaporation to dryness to obtain the required product, the applicants have found that more environmentally friendly solvents, in particular toluene or a mixture of toluene and n-heptane may be used in this stage and the product isolated by addition of a anti-solvent such as heptane, making the process much easier to perform.

Compounds of formula (V) are known compounds or may be prepared from known compounds by conventional methods.

Compounds of formula (IV) may be prepared in various ways for example as illustrated in WO2008/099145.

In a particular embodiment, the compound of formula (IV) is prepared by reacting a compound of formula (VI)

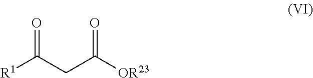

where R¹ is as defined above and R²³ is an alkyl group such as C₁₋₄alkyl, in particular ethyl; with a compound of formula (VII)

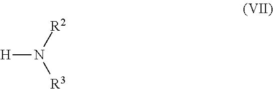

The reaction is suitably effected in an organic solvent such as toluene or xylene, at an elevated temperatures for example in the range of from 100 to 110° C. The compound of formula (IV) is suitably isolated by addition of a suitable anti-solvent, such as n-heptane. The reaction of compounds of formula (VI) with compounds of formula (VII) is novel and forms a further aspect of the invention. It is advantageous over previous processes for the production of compounds of formula (IV) since it avoids operations such as evaporation to dryness and the use of halocarbon reagents such as dichloromethane.

Compounds of formula (VII) are suitably generated in situ in the solvent by addition of a base to a solution of a salt, for example an acid addition salt such as a hydrochloride salt of a compound of formula (VII).

The reactions described above may be performed under standard conditions known to the person skilled in the art. The intermediates described above are commercially available, are known in the art or may be prepared by known procedures and/or by the procedures shown above.

It will be appreciated that certain of the various substituents in the compounds of the present invention may be introduced by standard aromatic substitution reactions or is generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example hydroxylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

In this specification the term “alkyl” includes both straight and branched chain alkyl groups but references to individual alkyl groups such as “propyl” are specific for the straight chain version only. For example, “C₁₋₄alkyl” includes propyl, isopropyl and t-butyl. However, references to individual alkyl groups such as ‘propyl’ are specific for the straight chain version only and references to individual branched chain alkyl groups such as ‘isopropyl’ are specific for the branched chain version only. A similar convention applies to other radicals therefore “C₁₋₄alkoxyC₁₋₄alkyl” would include 1-(C₁₋₄alkoxy)propyl, 2-(C₁₋₄alkoxy)ethyl and 3-(C₁₋₄alkoxy)butyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

A 4-7 membered saturated ring (for example formed between R^{5′} and R^{5″} and the nitrogen atom to which they are attached) is a monocyclic ring containing the nitrogen atom as the only ring atom.

“Heteroaryl”, unless otherwise specified, is a totally unsaturated, monocyclic ring containing 5 or 6 atoms of which at least 1, 2 or 3 ring atoms are independently chosen from nitrogen, sulphur or oxygen, which may, unless otherwise specified, be carbon-linked. A ring nitrogen atom may be optionally oxidised to form the corresponding N-oxide. Examples and suitable values of the term “heteroaryl” are thienyl, furyl, thiazolyl, pyrazolyl, isoxazolyl, imidazolyl, pyrrolyl, thiadiazolyl, isothiazolyl, triazolyl, pyrimidyl, pyrazinyl, pyridazinyl and pyridyl. Particularly “heteroaryl” refers to thienyl, furyl, thiazolyl, pyridyl, imidazolyl or pyrazolyl.

“Heterocycyl” is a 4-7 saturated, monocyclic ring having 1-3 ring heteroatoms selected from nitrogen, oxygen and sulphur. The ring sulphur may be optionally oxidised to SO₂.

A C₃₋₇cycloalkyl ring is a saturated carbon ring containing from 3 to 7 ring atoms.

A C₃₋₄cycloalkandiyl ring is a saturated carbon ring containing 3 or 4 ring atoms. It is a diradical with the radicals on different ring carbon atoms.

A C₃₋₄cycloalkanylidene ring is a saturated carbon ring containing 3 or 4 ring atoms. It is a diradical with the radicals on the same ring carbon atom.

A polycycloalkyl ring is a ring system in which either at least 2 rings are fused together or in which 2 ring have one ring atom in common (spiro).

A “saturated mono, bicyclic or bridged ring system optionally containing 1 or 2 additional ring heteroatoms selected from nitrogen, oxygen and sulphur”, unless otherwise specified contains 4-14 ring atoms. Particularly a mono ring contains 4-7 ring atoms, a bicyclic ring 6-14 ring atoms and a bridged ring system 6-14 ring atoms. Examples of mono rings include piperidinyl, piperazinyl and morpholinyl. Examples of bicyclic rings include decalin and 2,3,3a,4,5,6,7,7a-octahydro-1H-indene.

Bridged ring systems are ring systems in which there are two or more bonds common to two or more constituent rings. Examples of bridged ring systems include 1,3,3-trimethyl-6-azabicyclo[3.2.1]octane, 2-aza-bicyclo[2.2.1]heptane and 7-azabicyclo(2,2,1)heptane, 1- and 2-adamantanyl.

A “saturated, partially saturated or unsaturated monocyclic ring” is, unless otherwise specified, a 4-7 membered ring. Examples include, cyclopropyl, cyclobutyl, cyclopentyl, cyclopentenyl, cyclohexyl, cyclohexenyl and phenyl.

Examples of a “saturated or partially-saturated 5- or 6-membered ring optionally containing 1, 2 or 3 heteroatoms independently selected from nitrogen, oxygen and sulphur” include piperidinyl, piperazinyl and morpholinyl.

Examples of “C₁₋₄alkoxy” include methoxy, ethoxy and propoxy. Examples of “C₁₋₄alkoxyC₁₋₄alkyl” include methoxymethyl, ethoxymethyl, propoxymethyl, 2-methoxyethyl, 2-ethoxyethyl and 2-propoxyethyl. Examples of “C₁₋₄alkylS(O)ₙ wherein n is 0 to 2” include methylthio, ethylthio, methylsulphinyl, ethylsulphinyl, mesyl and ethylsulphonyl. Examples of “C₁₋₄alkylS(O)_{q}C₁₋₄alkyl” wherein q is 0 to 2″ include methylthio, ethylthio, methylsulphinyl, ethylsulphinyl, mesyl, ethylsulphonyl, methylthiomethyl, ethylthiomethyl, methylsulphinylmethyl, ethylsulphinylmethyl, mesylmethyl and ethylsulphonylmethyl. Examples of “C₁₋₄alkanoyl” include propionyl and acetyl. Examples of “N—(C₁₋₄alkyl)amino” include methylamino and ethylamino. Examples of “N,N—(C₁₋₄alkyl)₂-amino” include N,N-dimethylamino, N,N-diethylamino and N-ethyl-N-methylamino. Examples of “C₂₋₄alkenyl” are vinyl, allyl and 1-propenyl. Examples of “C₂₋₄alkynyl” are ethynyl, 1-propynyl and 2-propynyl. Examples of “N—(C₁₋₄alkyl)carbamoyl” are methylaminocarbonyl and ethylaminocarbonyl. Examples of “N,N—(C₁₋₄alkyl)₂-carbamoyl” are dimethylaminocarbonyl and methylethylaminocarbonyl. Examples of “C₃₋₇cycloalkylC₁₋₃alkalkyl” include cyclopropymethyl, 2-cyclopropylethyl, cyclobutylmethyl, cyclopentylmethyl and cyclohexylmethyl. Examples of “C₃₋₇cycloalkylC₂₋₃alkalkenyl” include 2-cyclopropylethenyl, 2-cyclopentylethenyl and 2-cyclohexylethenyl. Examples of “C₃₋₇cycloalkylC₂₋₃alkalkynyl” include 2-cyclopropylethynyl, 2-cyclopentylethynyl and 2-cyclohexylethynyl.

Examples of “C₃₋₇cycloalkyl(CH₂)ₘ-” include cyclopropymethyl, 2-cyclopropylethyl, cyclobutylmethyl, cyclopentylmethyl and cyclohexylmethyl. Examples of C₆₋₁₂polycycloalkyl(CH₂)ₘ— include norbornyl bicyclo[2.2.2]octane(CH₂)ₘ—, bicyclo[3.2.1]octane(CH₂)ₘ— and 1- and 2-adamantanyl(CH₂)ₘ—.

A suitable pharmaceutically-acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically-acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Some compounds of the formula (1) may have chiral centres and/or geometric isomeric centres (E- and Z-isomers), and it is to be understood that the invention encompasses the preparation of all such optical, diastereoisomers and geometric isomers that possess 11βHSD1 inhibitory activity.

It is also to be understood that certain compounds of the formula (1) can exist in solvated as well as unsolvated forms such as, for example, hydrated forms. It is to be understood that the invention encompasses the production all such solvated forms, which possess 11βHSD1 inhibitory activity.

Particular examples of compounds of formula (I) are compounds of formula (IA):

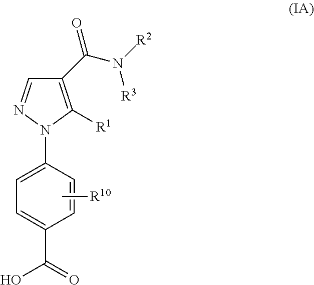

wherein R¹, R² and R³ are as hereinabove defined and R¹⁰ is selected from hydrogen, C₁₋₄alkyl, trifluoromethyl, C₁₋₄alkoxy and C₁₋₄alkylS-. In another aspect R¹⁰ is selected from hydrogen, methyl, trifluoromethyl, methoxy and methylthio. In another aspect R¹⁰ is hydrogen.

Particular values of variable groups in compounds of formula (I) are as follows. Such values may be used where appropriate with any of the definitions, claims or embodiments defined hereinbefore or hereinafter, for compounds of formula (1). The definitions of R¹, R² and R³ and variables within those groups may be used for the compound of formula (IA):

### Definition of R¹

a) In one aspect R¹ is C₃₋₆cycloalkyl optionally substituted by 1, 2 or 3 substituents independently selected from C₁₋₃alkyl, hydroxy, halo, oxo, cyano, fluoro, trifluoromethyl and C₁₋₃alkoxy.
b) In another aspect R¹ is C₃₋₆cycloalkyl.
c) In another aspect R¹ is C₃₋₆cycloalkylC₁₋₂alkyl optionally substituted by 1, 2 or 3 substituents independently selected from C₁₋₃alkyl, hydroxy, halo, oxo, cyano, fluoro, trifluoromethyl and C₁₋₃alkoxy.
d) In another aspect R¹ is C₃₋₄cycloalkylC₁₋₂alkyl.
e) In another aspect R¹ is C₁₋₄alkyl optionally substituted by 1, 2 or 3 substituents independently selected from C₁₋₃alkyl, hydroxy, halo, oxo, cyano, trifluoromethyl and C₁₋₃alkoxy.
f) In another aspect R¹ is C₁₋₄alkyl.
g) In another aspect R¹ is propyl optionally substituted by 1 or 2 substituents independently selected from C₁₋₃alkyl, hydroxy, halo, oxo, cyano, trifluoromethyl and C₁₋₃alkoxy.
h) In another aspect R¹ is tert-butyl

### Definition of R²

a) In one aspect, R² is selected from C₃₋₇cycloalkyl(CH₂)ₘ—, and C₆₋₁₂polycycloalkyl(CH₂)ₘ— (wherein m is 0, 1 or 2 and the rings are optionally substituted by 1, 2 or 3 substituents independently selected from R⁶) wherein m is 0, 1 or 2.
b) In another aspect, R² is selected from C₅₋₇cycloalkyl(CH₂)ₘ— and C₈₋₁₂polycycloalkyl(CH₂)ₘ— (wherein the rings are optionally substituted by 1, 2 or 3 substituents independently selected from R⁶) and wherein m is 0, 1 or 2.
c) In another aspect, R² is selected from C₅₋₇cycloalkyl(CH₂)ₘ—, C₇₋₁₀bicycloalkyl(CH₂)ₘ— and C₁₀tricycloalkyl(CH₂)ₘ— (wherein the cycloalkyl, bicycloalkyl and tricycloalkyl rings are optionally substituted by 1, 2 or 3 substituents independently selected from R⁶) and wherein m is 0, 1 or 2.
d) In yet another aspect, R² is selected from C₅₋₇cycloalkyl(CH₂)ₘ—, C₇₋₁₀bicycloalkyl(CH₂)ₘ— and adamantyl (wherein the cycloalkyl, bicycloalkyl and tricycloalkyl rings are optionally substituted by 1, 2 or 3 substituents independently selected from R⁶) and wherein m is 0, 1 or 2.
e) In yet another aspect, R² is adamantly.

### Definition of m

a) In one aspect, m is 0 or 1.
b) In another aspect, m is 0.

### Definition of R³

a) In one aspect, R³ is C₁₋₄alkyl.
b) In another aspect, R³ is hydrogen, methyl or ethyl.
c) In another aspect, R³ is hydrogen.
d) In another aspect, R³ is methyl.
e) In another aspect, R³ is ethyl.
f) In another aspect, R³ is cyclopropyl.

### Definition of R² and R³ Together

a) In another aspect, R² and R³ together with the nitrogen atom to which they are attached form a saturated 5 or 6-membered mono, 6-12 membered bicyclic or 6-12 membered bridged ring system optionally containing 1 or 2 additional ring heteroatoms selected from nitrogen, oxygen and sulphur and which is optionally fused to a saturated, partially-saturated or aryl monocyclic ring wherein the resulting ring system is optionally substituted by 1, 2, or 3 substituents independently selected from R⁷.

### Definition of R⁶

a) In one aspect, R⁶ is independently selected from hydroxyl, R⁹O—, R⁹CO— and R⁹C(O)O—
wherein R⁹ is as hereinabove defined.
b) In another aspect, R⁶ is independently selected from hydroxyl, R⁹O—, R⁹CO— and R⁹C(O)O—
wherein R⁹ is C₁₋₃alkyl optionally substituted by C₁₋₄alkoxy or carboxy.
c) In another aspect, R⁶ is independently selected from R⁹CON(R^{9′})—, R⁹SO₂N(R^{9″})- and (R^{9′})(R^{9″})NC(O)N(R^{9″})—;
wherein R⁹ is as hereinabove defined.
d) In another aspect, R⁶ is independently selected from R⁹CON(R^{9′})—, R⁹SO₂N(R^{9″})- and (R^{9′})(R^{9″})NC(O)N(R^{9″})—;
R⁹ is C₁₋₃alkyl optionally substituted by C₁₋₄alkoxy or carboxy;
R^{9′}, R^{9″} and R^{9″} are independently selected from hydrogen and C₁₋₃alkyl optionally substituted by C₁₋₄alkoxy or carboxy).
e) In another aspect, R⁶ is independently selected from (R^{9′})(R^{9″})NC(O)— and (R^{9′})(R^{9″})N—;
wherein R^{9′} and R^{9″} are as hereinabove defined.
f) In another aspect, R⁶ is independently selected from (R^{9′})(R^{9″})NC(O)— and (R^{9′})(R^{9″})N—;
wherein R^{9′} and R^{9″} are independently selected from hydrogen and C₁₋₃alkyl optionally substituted by C₁₋₄alkoxy or carboxy.
g) In one aspect R⁶ is selected from methyl, trifluoromethyl, chloro, fluoro, bromo, methoxy, ethoxy, trifluormethoxy, methanesulfonyl, ethanesulfonyl, methylthio, ethylthio, amino, N-methylamino, N-ethylamino, N-propylamino, N,N-dimethylamino, N,N-methylethylamino or N,N-diethylamino.
h) In another aspect, R⁶ is optionally substituted phenyl, pyridyl or pyrimidyl.
i) In another aspect, R⁶ is optionally substituted pyrid-2-yl, pyrid-3-yl or pyrid-4-yl.

### Definition of R⁷

a) In another aspect, R⁷ is independently selected from hydroxyl, halo, oxo, cyano, trifluoromethyl, R⁹ and R⁹O— (wherein R⁹ is as hereinabove defined).
b) In another aspect, R⁷ is independently selected from hydroxyl, halo, trifluoromethyl, R⁹ and R⁹O— (wherein R⁹ is as hereinabove defined).

### Definition of R⁹

a) In one aspect, R⁹ is independently selected from C₁₋₃alkyl.

### Definition of R^{9′}, R^{9″} and R^{9′″}

a) In one aspect, R^{9′}, R^{9″} and R^{9′″} are independently selected from hydrogen and C₁₋₃alkyl.

### Definition of Y

a) In one aspect, Y is independently selected from direct bond, —CH₂— and —CH₂CH₂—.
b) In one aspect, Y is independently selected from —CH₂— and —CH₂CH₂—.
c) In another aspect Y is a direct bond.

### Definition of A

a) In one aspect A is phenyl optionally substituted by R¹⁰.
b) In another aspect A is heteroaryl optionally substituted by R¹⁰ and R¹¹.
c) In another aspect A is thienyl optionally substituted by R¹⁰ and R¹¹.
d) In another aspect A is pyridyl optionally substituted by R¹⁰ and R¹¹.
e) In another aspect A is phen-1,4-diyl

### Definition of R¹⁰

a) In one aspect, R¹⁰ is independently selected from C₁₋₄alkyl, hydroxyl, cyano, trifluoromethyl, trifluoromethoxy, difluoromethoxy, halo, C₁₋₄alkoxy and C₁₋₄alkoxyC₁₋₄alkyl.
b) In another aspect, R¹⁰ is independently selected from methyl, ethyl, hydroxyl, cyano, trifluoromethyl, trifluoromethoxy, difluoromethoxy, halo, methoxy, ethoxy, methoxymethyl and ethoxymethyl.
c) In another aspect, R¹⁰ is independently selected from methyl, ethyl, cyano, trifluoromethyl, trifluoromethoxy, difluoromethoxy, halo, methoxy, ethoxy.

### Definition of R¹¹

a) In one aspect, R¹¹, is independently selected from C₁₋₃alkyl, trifluoromethyl and difluoromethyl.
b) In one aspect, R¹¹, is independently selected from methyl, ethyl, trifluoromethyl and difluoromethyl.

### Definition of X

a) In one aspect, X is independently selected from direct bond, —CH₂—, —CHMe—, —CMe₂-, —CH₂CH₂—, —CH₂O— and —CH₂S—.
b) In one aspect, X is independently selected from —CH₂—, —CHMe—, —CMe₂-, —CH₂CH₂—, —CH₂O— and —CH₂S—.
c) In another aspect X is independently selected from cyclopropanylidene, cyclobutanylidene, cyclopropane-1,2-diyl and cyclobutan-1,2-diyl.
d) In another aspect X is a direct bond.

In one aspect, R¹², R¹³, R¹⁴, R¹⁵, R¹⁶, R¹⁷, R¹⁸, R¹⁹, R²⁰ and R²¹ are hydrogen.

In one aspect R¹ is optionally substituted by 0 substituents.

In one aspect R¹ is optionally substituted by 1 substituent.

In one aspect R¹ is optionally substituted by 2 substituents.

In one aspect R¹ is optionally substituted by 3 substituents.

In one aspect R² is optionally substituted by 0 substituents.

In one aspect R² is optionally substituted by 1 substituent.

In one aspect R² is optionally substituted by 2 substituents.

In one aspect R² is optionally substituted by 3 substituents.

In one aspect R³ is optionally substituted by 0 substituents.

In one aspect R³ is optionally substituted by 1 substituent.

In one aspect R³ is optionally substituted by 2 substituents.

In one aspect R³ is optionally substituted by 3 substituents.

In one aspect the group formed by R² and R³ together is optionally substituted by 0 substituents.

In one aspect the group formed by R² and R³ together is optionally substituted by 1 substituent.

In one aspect the group formed by R² and R³ together is optionally substituted by 2 substituents.

In one aspect the group formed by R² and R³ together is optionally substituted by 3 substituents.

In one aspect A is optionally substituted by 0 substituents.

In one aspect A is optionally substituted by 1 substituent.

In one aspect A is optionally substituted by 2 substituents.

In one aspect A is optionally substituted by 3 substituents.

In one aspect the phenyl and heteroaryl groups in R⁶ and R⁷ are independently optionally substituted by 0 substituents.

In one aspect the phenyl and heteroaryl groups in R⁶ and R⁷ are independently optionally substituted by 1 substituent.

In one aspect the phenyl and heteroaryl groups in R⁶ and R⁷ are independently are optionally substituted by 2 substituents.

In one aspect the phenyl and heteroaryl groups in R⁶ and R⁷ are independently are optionally substituted by 3 substituents.

In another aspect the invention relates to a process for preparing 4-[4-(2-adamantylcarbamoyl)-5-tert-butyl-pyrazol-1-yl] benzoic acid or a pharmaceutically-acceptable salt thereof, which process comprises the step of reacting a compound of the formula (IIB):

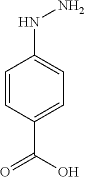

or salt thereof;

with a compound of formula (IIIB):

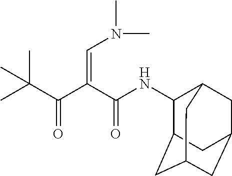

and thereafter if necessary or desirable carrying out one or more of the following steps:

i) forming a pharmaceutically-acceptable salt thereof; and

ii) purifying the product.

In a particular aspect, the aryl hydrazine is a hydrochloride salt.

A particular example of a compound of formula (I) is 4-[4-(2-adamantyl-carbamoyl)-5-tert-butyl-pyrazol-1-yl] benzoic acid. In one aspect this is prepared in a pure polymorphic form. In particular, this compound is in a crystalline form (referred to herein as ‘Form 4’), which has an X-ray powder diffraction pattern with a peak at about 2-theta=16.2°, when measured using CuKa radiation. Suitably the compound is in a crystalline form which has an X-ray powder diffraction pattern with at least two specific peaks at about 2-theta=16.2° and 20.6°, for example with specific peaks at about 2-theta=16.2, 20.6 and 17.7°, more particularly with specific peaks at about 2-theta=16.2, 20.6, 17.7, is 10.8 and 15.5° and yet more particularly with specific peaks at about 2-theta=16.2, 20.6, 17.7, 10.8, 15.5, 20.9, 26.1, 11.6, 26.7 and 18.1°, wherein any of said values may be plus or minus 0.5° 2-theta.

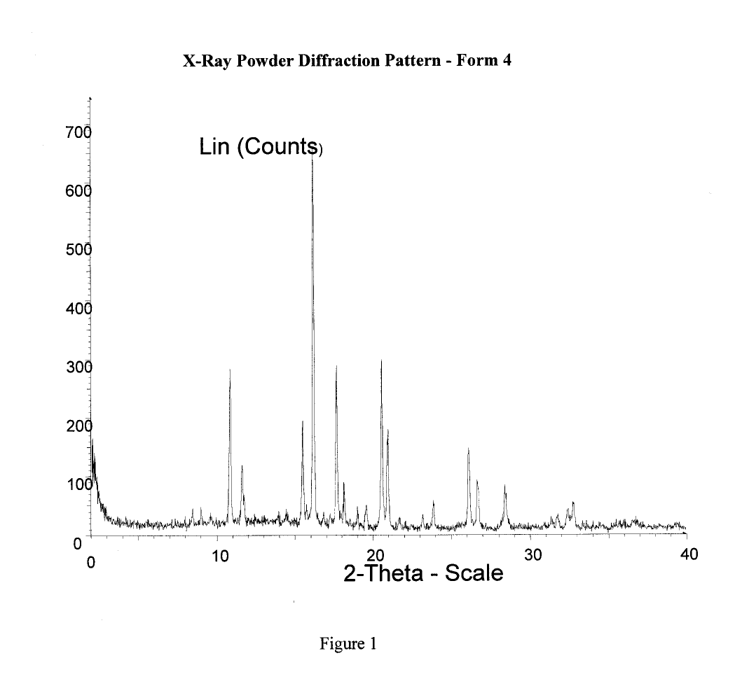

For instance, the compound is in a crystalline form which has an X-ray powder diffraction pattern, using CuKa radiation, substantially the same as the X-ray powder diffraction pattern shown in FIG. 1.

TABLE C
Ten most Prominent X-Ray Powder Diffraction
peaks Form 4 of the Agent

| Angle 2-Theta (2θ) | Relative Intensity |
|---|---|
| 16.2 | vs |
| 20.6 | s |
| 17.7 | s |
| 10.8 | s |
| 15.5 | m |
| 20.9 | m |
| 26.1 | m |
| 11.6 | m |
| 26.7 | m |
| 18.1 | m |

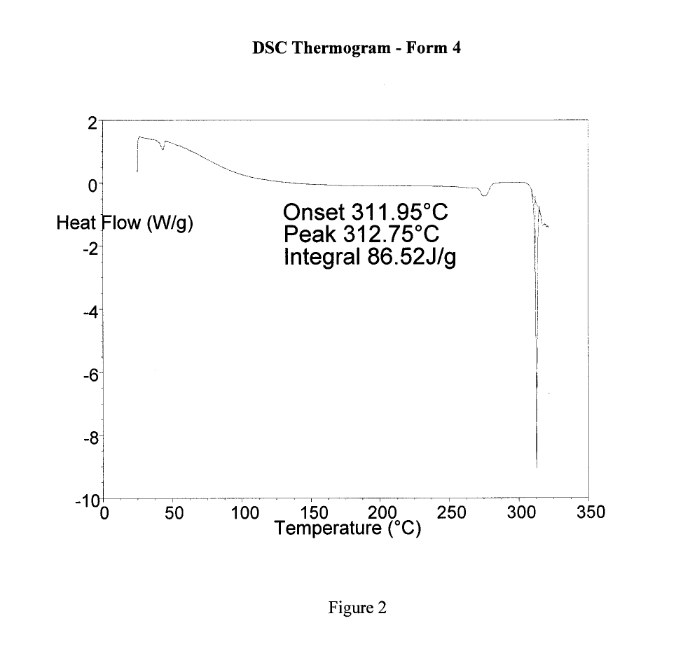

DSC analysis of Form 4 shows a peak at 262.0° C. followed by a subsequent melt with an onset of 312.0° C. The DSC thermogram of form 4 is depicted in FIG. 2. Such forms are obtainable using the process exemplified hereinafter.

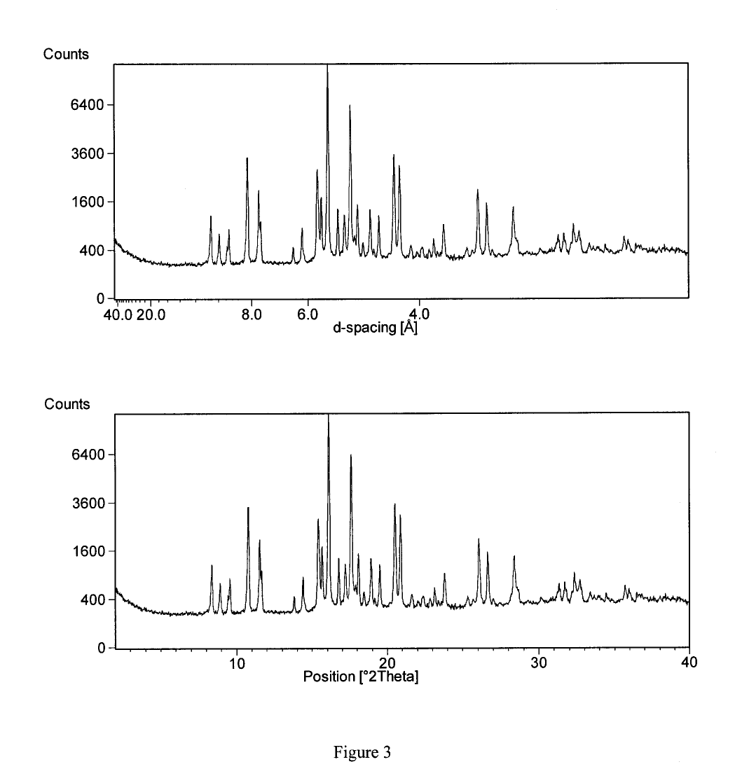

Another more pure sample of form 4 gave the XPRD pattern shown in FIG. 3. The position of d-spacing are shown in table D.

TABLE D
d-Spacing for form 4
d-spacing [Å]

8.2
7.7
5.8
5.6
5.1
4.76
4.40
4.32
3.50
3.44

Another aspect of the invention relates to the preparation of 4-[4-(2-adamantyl-carbamoyl)-5-tert-butyl-pyrazol-1-yl] benzoic acid as form 4 by recrysallisation of 4-[4-(2-adamantyl-carbamoyl)-5-tert-butyl-pyrazol-1-yl] benzoic acid as form 1 from THF, water and acetonitrile.

Yet another aspect of the invention relates to the preparation of 4-[4-(2-adamantyl-carbamoyl)-5-tert-butyl-pyrazol-1-yl] benzoic acid as form 4 by recrysallisation of 4-[4-(2-adamantyl-carbamoyl)-5-tert-butyl-pyrazol-1-yl] benzoic acid as form 1 from THF and acetonitrile.

Yet another aspect of the invention relates to the preparation of 4-[4-(2-adamantyl-carbamoyl)-5-tert-butyl-pyrazol-1-yl] benzoic acid as form 4 by recrysallisation of 4-[4-(2-adamantyl-carbamoyl)-5-tert-butyl-pyrazol-1-yl] benzoic acid as form 1 from DMF and acetonitrile.

Yet another aspect of the invention relates to the preparation of 4-[4-(2-adamantyl-carbamoyl)-5-tert-butyl-pyrazol-1-yl] benzoic acid as form 4 by recrysallisation of 4-[4-(2-adamantyl-carbamoyl)-5-tert-butyl-pyrazol-1-yl] benzoic acid as form 1 from acetic acid and acetonitrile.

Yet another aspect of the invention relates to the preparation of 4-[4-(2-adamantyl-carbamoyl)-5-tert-butyl-pyrazol-1-yl] benzoic acid as form 4 by recrysallisation of 4-[4-(2-adamantyl-carbamoyl)-5-tert-butyl-pyrazol-1-yl] benzoic acid as form 1 from 2-methylTHF and acetonitrile.

Another aspect of the invention relates to the preparation of 4-[4-(2-adamantyl-carbamoyl)-5-tert-butyl-pyrazol-1-yl] benzoic acid as form 4 by recrysallisation of 4-[4-(2-adamantyl-carbamoyl)-5-tert-butyl-pyrazol-1-yl] benzoic acid as a hydrate from THF, water and acetonitrile.

Yet another aspect of the invention relates to the preparation of 4-[4-(2-adamantyl-carbamoyl)-5-tert-butyl-pyrazol-1-yl] benzoic acid as form 4 by recrystallisation of 4-[4-(2-adamantyl-carbamoyl)-5-tert-butyl-pyrazol-1-yl] benzoic acid as a hydrate from THF and acetonitrile.

Yet another aspect of the invention relates to the preparation of 4-[4-(2-adamantyl-carbamoyl)-5-tert-butyl-pyrazol-1-yl] benzoic acid as form 4 by recrysallisation of 4-[4-(2-adamantyl-carbamoyl)-5-tert-butyl-pyrazol-1-yl] benzoic acid as a hydrate from DMF and acetonitrile.

Yet another aspect of the invention relates to the preparation of 4-[4-(2-adamantyl-carbamoyl)-5-tert-butyl-pyrazol-1-yl] benzoic acid as form 4 by recrysallisation of 4-[4-(2-adamantyl-carbamoyl)-5-tert-butyl-pyrazol-1-yl] benzoic acid as a hydrate from acetic acid and acetonitrile.

Yet another aspect of the invention relates to the preparation of 4-[4-(2-adamantyl-carbamoyl)-5-tert-butyl-pyrazol-1-yl] benzoic acid as form 4 by recrysallisation of 4-[4-(2-adamantyl-carbamoyl)-5-tert-butyl-pyrazol-1-yl] benzoic acid a hydrate from 2-methylTHF and acetonitrile.

Yet another aspect of the invention relates to a crystalline form of 4-[4-(2-adamantylcarbamoyl)-5-tert-butyl-pyrazol-1-yl]benzoic acid having an X-ray diffraction pattern with peaks at the following d-spacing values: 14.3 and 5.5 Å.

Yet another aspect of the invention relates to a crystalline form of 4-[4-(2-adamantylcarbamoyl)-5-tert-butyl-pyrazol-1-yl]benzoic acid having an X-ray diffraction pattern with peaks at the following d-spacing values: 14.3, 5.5, 13.1 and 7.2 Å

Yet another aspect of the invention relates to a crystalline form of 4-[4-(2-adamantylcarbamoyl)-5-tert-butyl-pyrazol-1-yl]benzoic acid having an X-ray diffraction pattern with peaks at the following d-spacing values: 14.3, 5.5, 13.1, 7.2 and 5.0 Å.

Yet another aspect of the invention relates to a crystalline form of 4-[4-(2-adamantylcarbamoyl)-5-tert-butyl-pyrazol-1-yl]benzoic acid having an X-ray diffraction pattern with peaks at the following d-spacing values: 5.5 and 14.4 Å.

Yet another aspect of the invention relates to a crystalline form of 4-[4-(2-adamantylcarbamoyl)-5-tert-butyl-pyrazol-1-yl]benzoic acid having an X-ray diffraction pattern with peaks at the following d-spacing values: 5.5, 14.4, 16.7 and 5.1 Å.

Yet another aspect of the invention relates to a crystalline form of 4-[4-(2-adamantylcarbamoyl)-5-tert-butyl-pyrazol-1-yl]benzoic acid having an X-ray diffraction pattern with peaks at the following d-spacing values: 5.5, 14.4, 16.7, 5.1, 13.1 and 12.8 Å

Yet another aspect of the invention relates to a crystalline form of 4-[4-(2-adamantylcarbamoyl)-5-tert-butyl-pyrazol-1-yl]benzoic acid having an X-ray diffraction pattern with peaks at the following d-spacing values: 8.1 and 13.2 Å,

Yet another aspect of the invention relates to a crystalline form of 4-[4-(2-adamantylcarbamoyl)-5-tert-butyl-pyrazol-1-yl]benzoic acid having an X-ray diffraction pattern with peaks at the following d-spacing values: 8.1, 13.2, 17.9 and 5.9 Å

Yet another aspect of the invention relates to a crystalline form of 4-[4-(2-adamantylcarbamoyl)-5-tert-butyl-pyrazol-1-yl]benzoic acid having an X-ray diffraction pattern with peaks at the following d-spacing values: 8.1, 13.2, 17.9, 5.9, 6.4 and 5.0 Å.

Yet another aspect of the invention relates to a crystalline form of 4-[4-(2-adamantylcarbamoyl)-5-tert-butyl-pyrazol-1-yl]benzoic acid having an X-ray diffraction pattern with peaks at the following d-spacing values: 14.2 and 10.1 Å.

Yet another aspect of the invention relates to a crystalline form of 4-[4-(2-adamantylcarbamoyl)-5-tert-butyl-pyrazol-1-yl]benzoic acid having an X-ray diffraction pattern with peaks at the following d-spacing values: 14.2, 10.1, 7.1 and 6.4 Å

Yet another aspect of the invention relates to a crystalline form of 4-[4-(2-adamantylcarbamoyl)-5-tert-butyl-pyrazol-1-yl]benzoic acid having an X-ray diffraction pattern with peaks at the following d-spacing values: 14.2, 10.1, 7.1, 6.4, 5.6 and 4.50 Å

Yet another aspect of the invention relates to a crystalline form of 4-[4-(2-adamantylcarbamoyl)-5-tert-butyl-pyrazol-1-yl]benzoic acid having an X-ray diffraction pattern with peaks at the following d-spacing values: 4.71 and 6.3 Å

Yet another aspect of the invention relates to a crystalline form of 4-[4-(2-adamantylcarbamoyl)-5-tert-butyl-pyrazol-1-yl]benzoic acid having an X-ray diffraction pattern with peaks at the following d-spacing values: 4.71, 6.3, 7.6 and 4.12 Å

Yet another aspect of the invention relates to a crystalline form of 4-[4-(2-adamantylcarbamoyl)-5-tert-butyl-pyrazol-1-yl]benzoic acid having an X-ray diffraction pattern with peaks at the following d-spacing values: 12.7 and 5.7 Å

Yet another aspect of the invention relates to a crystalline form of 4-[4-(2-adamantylcarbamoyl)-5-tert-butyl-pyrazol-1-yl]benzoic acid having an X-ray diffraction pattern with peaks at the following d-spacing values: 12.7, 5.7, 16.3 and 5.5 Å.

Yet another aspect of the invention relates to a crystalline form of 4-[4-(2-adamantylcarbamoyl)-5-tert-butyl-pyrazol-1-yl]benzoic acid having an X-ray diffraction pattern with peaks at the following d-spacing values: 12.7, 5.7, 16.3, 5.5 and 4.23 Å.

The invention is illustrated by the following examples.

### ABBREVIATIONS USED IN EXAMPLES

DCM=Di Chloro Methane

DMF=Di-Methyl Formamid

MIBK=Methyl Iso-Buthyl Keton

MTBE=Methyl Tert-Buthyl Ether

TGA=Thermo Gravimetric Analysis

THF=Tetra Hydro Furane

XRPD=X-Ray Powder Diffraction

### STEP-1

### Example 1

### Synthesis of (2)-N-(2-adamantyl)-2-(dimethylaminomethylidene)-4,4-dimethyl-3-oxo-pentanamide

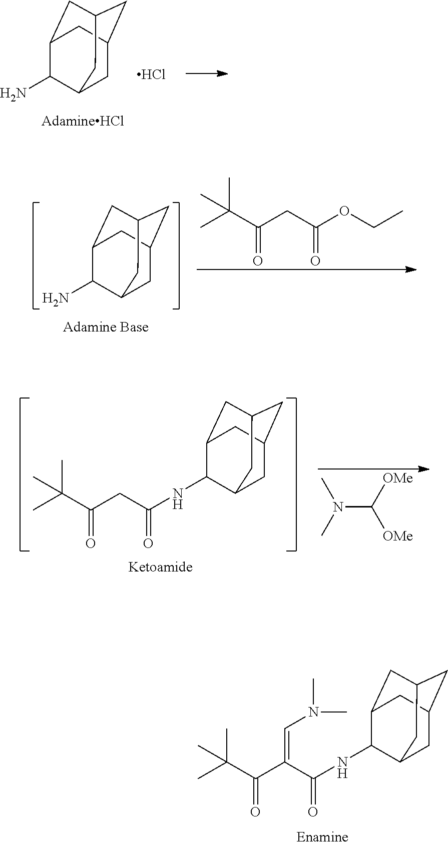

To a suspension of 2-adamantanamine hydrochloride (25.0 g, 0.13 mol) in water (75.0 ml, 3.0 rel. vol) was added toluene (100.0 ml, 4.0 rel. vol). A 10.0% w/w aqueous sodium hydroxide solution (1.25 mol. eq) was fed into the above solution and stirred for 10 to 15 minutes. The organic layer was separated and the aqueous layer re-extracted with toluene (75.0 ml, 3.0 rel. vol) and combined with the separated organic layer. The combined organic layer was washed with 5.0% w/w sodium chloride solution (75 ml, 3.0 rel. vol.) and separated. Ethyl pivaloylacetate (26.01 g, 0.15 mol) was added to the organic layer was the reaction mass heated to reflux at 110 to 112° C. The solvent (4 to 5 rel. vol.) was collected azeotropically over 4 to 5 hours. The reaction mass was cooled to 40 to 45° C. and n-heptane (200.0 ml, 8.0 rel. vol) added at 35 to 40° C. followed by DMF-DMA (26.45 g, 0.20 mol) and triethylamine (13.48 g, 0.13 mol) at 30 to 35° C. The reaction mass temperature was raised to 90 to 93° C. and maintained for 2 to 3 hours. The methanol generated as a by-product was collected azeotropically during the reaction. The reaction was cooled to 20 to 25° C. and stirred for 1.0 hr at that temperature. The precipitated product was filtered, bed washed with n-heptane (100.0 ml, 4.0 rel. vol) and the product dried under vacuum (50-100 mbar) at 35-40° C. for 3 to 4 hours to give (2)-N-(2-adamantyl)-2-(dimethylaminomethylidene)-4,4-dimethyl-3-oxo-pentanamide (Yield, 86%). The product was packed under nitrogen atmosphere and stored below 10° C. as it was found to be unstable at room temperature.

### Example-2

To a suspension of 2-adamantanamine hydrochloride (25.0 g, 0.13 mol) in water (75.0 ml, 3.0 rel. vol) was added toluene (100.0 ml, 4.0 rel. vol). A 10.0% w/w aqueous sodium hydroxide solution (1.25 mol. eq) was fed into the above solution and stirred for 10 to 15 minutes. The organic layer was separated and the aqueous layer re-extracted with toluene (75.0 ml, 3.0 rel. vol) and combined with the separated organic layer. The combined organic layer was washed with 5.0% w/w sodium chloride solution (75 ml, 3.0 rel. vol.) and separated. Ethyl pivaloylacetate (26.01 g, 0.15 mol) was added to the organic layer was the reaction mass heated to reflux at 110 to 112° C. The solvent (4 to 5 rel. vol.) was collected azeotropically over 4 to 5 hours. The reaction mass was cooled to 40 to 45° C. and n-heptane (200.0 ml, 8.0 rel. vol) added at 35 to 40° C. followed by DMF-DMA (26.45 g, 0.20 mol) at the same temperature. The reaction mass temperature was raised to 85 to 90° C. and maintained for 4 to 5 hours. The methanol generated as a by-product was collected azeotropically during the reaction. The reaction was cooled to 20 to 25° C. and stirred for 1.0 hr at that temperature. The precipitated product was filtered, bed washed with n-heptane (100.0 ml, 4.0 rel. vol) and the product dried under vacuum (50-100 mbar) at 35-40° C. for 3 to 4 hours to give (2)-N-(2-adamantyl)-2-(dimethylaminomethylidene)-4,4-dimethyl-3-oxo-pentanamide (Yield, 72%). The product was packed under nitrogen atmosphere and stored below 10° C. as it was found to be unstable at room temperature.

### Chromatographic Conditions:—

Sunfire C18, 150×4.6 mm, 5μ, mobile phase used is di-sodium hydrogen phosphate buffer using methanol as organic solvent, 1.0 mL/min flow rate, injection volume is 204, run time is 20 minutes using refractive index detector.

### Retention Times:

N-(2-adamantyl)-4,4-dimethyl-3-oxo-pentanamide RT: 11.0 min
(2)-N-(2-adamantyl)-2-(dimethylaminomethylidene)-4,4-dimethyl-3-oxo-pentanamide RRT: 1.18 min.

1H NMR (400.13 MHz, DMSO-d6) δ 1.13 (9H, s), 1.47 (2H, d), 1.69-1.83 (10H, m), 2.03 (2H, d), 2.92 (6H, s), 3.90 (1H, d), 7.24 (1H, s), 7.94 (1H, d)

m/z (ESI+) (M+H)+=333

If necessary the N-(2-adamantyl)_{—}4,4-dimethyl-3-oxopentanamide intermediate may be isolated:

### Chromatographic Conditions: —

HP-5MS column, Helium as carrier gas, 1.0 mL/min flow rate, solvent delay up to 1.5 min, oven temperature=initial 50° C., hold for 2 min, and then ramping @20° C./min up to 280° C. and injection volume is 1.04.

### Retention Times:

2-Adamantanamine Hydrochloride RT 8.1 min

N-(2-adamantyl)-4,4-dimethyl-3-oxo-pentanamide RRT: 1.617 min.

1H NMR (400.13 MHz, DMSO-d6) δ 1.08-1.09 (9H, m), 1.50 (2H, d), 1.66-1.89 (10H, m), 1.95-2.00 (2H, m), 3.53 (1.4H, s), 3.80-3.94 (1H, m), 5.30 (0.3H, s), 7.77-7.87 (1H, m), 14.43 (0.3H, s) (2:1 mixture of keto and enol forms) m/z (ESI+) (M+H)+=278

### STEP-2

### Synthesis of 4-[4-(2-Adamantylcarbamoyl)-5-tert-butyl-pyrazol-1-yl]benzoic acid (Form-1)

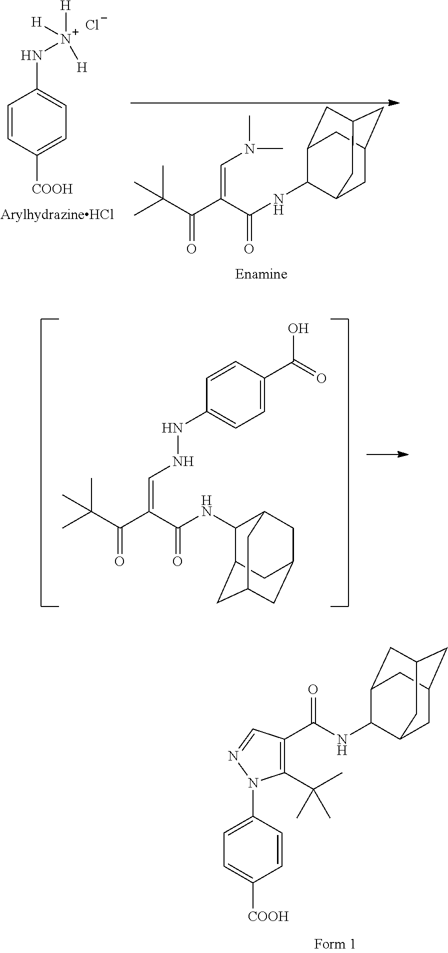

4-Hydrazinobenzoic acid.HCl (14.11 g, 0.075 mol), and (2)-N-(2-adamantyl)-2-(dimethylaminomethylidene)-4,4-dimethyl-3-oxo-pentanamide (25.0 g, 0.075 mol) were put into a jacketed reactor followed by isopropyl alcohol (315 ml, 12.6 rel. vol.) and water (35 ml, 1.4 rel. vol.). The reaction mass was stirred at 20 to 25° C. for about 45 to 60 minutes. The contents were heated to reflux at 78 to 80° C. and maintained at that temperature for 90 minutes. The reaction mass was cooled to 50 to 55° C. and then water (150 ml, 6 rel. vol.) added at the same temperature. The contents were further cooled to ambient temperature (20 to 25° C.) and stirred for 1.0 hour at the same temperature. The precipitated product was filtered and then washed with a mixture of 1:1 ratio of isopropyl alcohol:water (250 ml, 10.0 rel. vol.) to yield 4-[4-(2-adamantylcarbamoyl)-5-tert-butyl-pyrazol-1-yl] benzoic acid form 1. The product was dried under vacuum at 50 to 55° C. for 4 to 5 hours and used in the next step without further purification (Yield: 80%).

1H NMR (400.13 MHz, DMSO-d6) δ 1.19 (9H, s), 1.49 (2H, d), 1.70-1.96 (10H, m), 2.09 (2H, d), 3.98-4.01 (1H, m), 7.49-7.53 (2H, m), 7.61 (1H, s), 8.06-8.09 (2H, m), 8.20 (1H, d), 13.30 (1H, s)

m/z (ESI+) (M+H)+=422

m.p. 308.8° C. (onset)

### Chromatographic Conditions:—

Zorbax SB-Aq, 150×4.6 mm, 5μ, mobile phase used is formic acid buffer using acetonitrile as organic solvent, 1.0 mL/min flow rate, injection volume is 20 μL, run time is 18 minutes using UV detector wavelength 220,320 nm.

### Retention Times:

[((2)-N-(2-Adamantyl)-2-(dimethylaminomethylidene)-4,4-dimethyl-3-oxo-pentanamide RT 14.2 min

4-[4-(2-Adamantylcarbamoyl)-5-tert-butyl-pyrazol-1-yl] benzoic acid RRT 0.77 min (10.0 min)

Intermediate RRT 0.79 (11.2 min)

### STEP (3)

### Polymorphs Conversion (Form-1 to Form-4)

### 4-[4-(2-Adamantylcarbamoyl)-5-tert-butyl-pyrazol-1-yl]benzoic acid

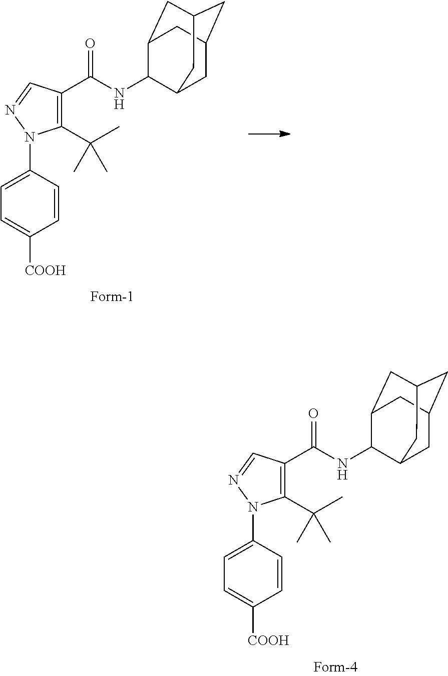

4-[4-(2-Adamantylcarbamoyl)-5-tert-butyl-pyrazol-1-yl] benzoic acid form 1 (20.0 g, 0.047 mol) followed by tetrahydrofuran (9.0 rel. vol) and water (0.5 rel vol) were added to a suitable jacketed reactor. The contents were stirred for 15 minutes, filtered through filter paper and washed with tetrahydrofuran (1.0 rel. vol). The combined filtrate was transferred to reactor and temperature of mass increased to 58 to 62° C. Acetonitrile (20.0 rel. vol) was added whilst maintaining the temperature at 55 to 65° C. The temperature of reaction mass was increased to 68±2° C. and maintained there for 22 hours. The contents were cooled to 20 to 25° C. and stirred for 2 hours. The product was filtered and the bed washed with acetonitrile (5.0 rel. vol). The wet cake was dried under vacuum (50-100 mbar) at 45 to 50° C. for 4 hours to yield polymorph form 4 (80%).

### Alternatively:

Tetrahydrofuran (9.0 rel. vol) and water (0.5 rel vol) were added to 4-[4-(2-adamantylcarbamoyl)-5-tert-butyl-pyrazol-1-yl] benzoic acid form 1 (20.0 g, 0.047 mol) and the mixture stirred for 15 minutes and then filtered through filter paper. The residue was washed with tetrahydrofuran (1.0 rel. vol) and the combined filtrate transferred to a reactor and the reaction temperature raised to 58 to 62° C. Acetonitrile (20.0 rel. vol) was added whilst maintaining the reaction at 55 to 65° C. The reaction temperature was raised to 68±2° C., maintained there for 22 hours, then cooled to 20 to 25° C. and stirred for 2 hours. The product was filtered and the bed washed with acetonitrile (5.0 rel. vol). The wet cake was dried under vacuum (50-100 mbar) at 45 to 50° C. for 4 hours to give polymorph 4 (yield 80%) as confirmed by XRPD.

### Alternatively:

Tetrahydrofuran (10.0 rel. vol) was added to 4-[4-(2-adamantylcarbamoyl)-5-tert-butyl-pyrazol-1-yl] benzoic acid form 1 (5.0 g, 0.012 mol) and the temperature raised to 58 to 62° C. Acetonitrile (20.0 rel. vol) was added whilst maintaining the reaction at 55 to 65° C. The temperature of the reaction was maintained at 68±2° C. for 20 hours. The contents were cooled to 20 to 25° C. and stirred for 2 hours. The product was filtered and the wet cake washed with acetonitrile (5.0 vol) and then dried in a vacuum oven (50-100 mbar) at 45 to 50° C. for 4 hours to give polymorph 4 (yield 90%) as confirmed by XRPD and Solid state NMR.

### Alternatively:

N,N-Dimethylformamide (5.0 rel. vol) and acetonitrile (5.0 vol) were added to 4-[4-(2-adamantylcarbamoyl)-5-tert-butyl-pyrazol-1-yl] benzoic acid form 1 (5.0 g, 0.012 mol) and the reaction temperature raised to 60 to 65° C. Acetonitrile (15.0 rel. vol) was added whilst maintaining the temperature at 55 to 65° C. The temperature of the reaction was raised to 75 to 78° C. and maintained there for 20 hours. The contents were cooled to 20 to 25° C. and stirred for 2 hours. The product was filtered and bed washed with acetonitrile (5.0 rel. vol) and then dried in a vacuum oven (50-100 mbar) at 45 to 50° C. for 4 hours to give polymorph 4 (yield 88%) as confirmed by XRPD.

### Alternatively:

Acetic acid (10.0 rel. vol) was added to 4-[4-(2-adamantylcarbamoyl)-5-tert-butyl-pyrazol-1-yl] benzoic acid form 1 (5.0 g, 0.012 mol) and the temperature raised to 75 to 78° C. Acetonitrile (20.0 rel. vol) was added whilst maintaining the temperature at 70 to 78° C. The mixture was stirred at 75 to 78° C. and maintained there for 22 hours. The contents were cooled to 20 to 25° C. and stirred for 2 hours. The product was filtered and the bed washed with acetonitrile (5.0 rel. vol) and then dried in a vacuum oven (50-100 mbar) at 45 to 50° C. for 4 hours to polymorph 4 (yield 66%) as confirmed by XRPD.

### Alternatively:

2-Methyl-THF (10.0 rel. vol) was added to 4-[4-(2-adamantylcarbamoyl)-5-tert-butyl-pyrazol-1-yl] benzoic acid form 1 (5.0 g, 0.012 mol) and the temperature raised to 70 to 75° C. Acetonitrile (20.0 rel. vol) was added whilst maintaining the temperature at 70 to 75° C. and then allowed to stir at 75 to 78° C. for 23 hours. The contents were cooled to 20 to 25° C. and stirred for 2 hours. The product was filtered and the bed washed with acetonitrile (5.0 rel. vol) and then dried in a vacuum oven (50-100 mbar) at 45 to 50° C. for 4 hours to give polymorph 4 (yield 93%) as confirmed by XRPD.

### Synthesis of 4-[4-(2-Adamantylcarbamoyl)-5-tert-butyl-pyrazol-1-yl]benzoic acid (Hydrate)

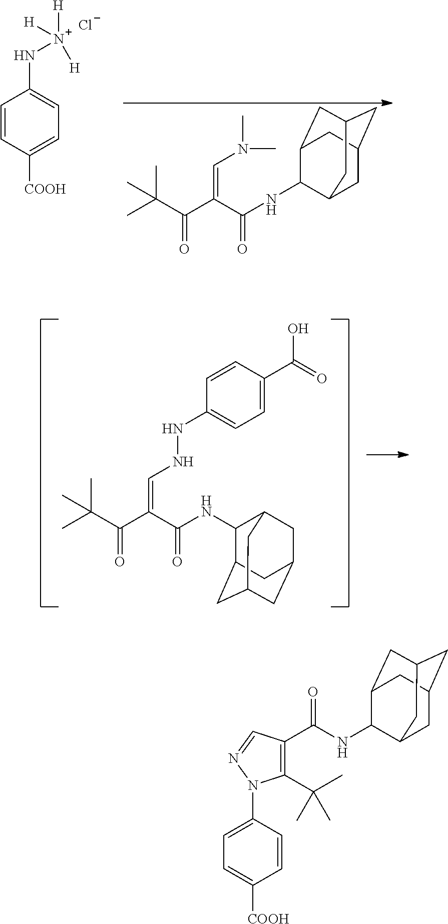

4-Hydrazinobenzoic acid.HCl (2.86 g, 0.010 mol), water (10 mL) and methanol (60 mL) were added to a suitable flask and then a solution of (2)-N-(2-adamantyl)-2-(dimethylaminomethylidene)-4,4-dimethyl-3-oxo-pentanamide (5.10 g, 0.010 mol) in methanol (30 mL) was added. The contents were stirred at 20-25° C. for 1 hour and then heated to reflux at 65-66° C. for 90 minutes. The reaction mass was cooled to 40 to 45° C. and then 25% aqueous NaOH solution (2.0 eq.) was added slowly and continued to stir at the same temperature for 60 minutes. The contents were further cooled to ambient temperature (20 to 25° C.) and 10% aqueous HCl (2.7 eq) was added slowly and continued to stir for 60 minutes at the same temperature. Charged water (50 mL), precipitated product was filtered and then washed the bed with water (25 mL) followed by a mixture of 1:1 ratio of methanol:water (50 mL) to yield 4-[4-(2-adamantylcarbamoyl)-5-tert-butyl-pyrazol-1-yl] benzoic acid (hydrate). The product was dried under vacuum at 50 to 55° C. for 4 to 5 hrs to give hydrated form as confirmed by XRPD (Yield: 86%).

1H NMR (400.13 MHz, DMSO-d6) δ 1.19 (9H, s), 1.49 (2H, d), 1.70-1.96 (10H, m), 2.09 (2H, d), 3.98-4.01 (1H, m), 7.49-7.53 (2H, m), 7.61 (1H, s), 8.06-8.09 (2H, m), 8.20 (1H, d)

m/z (ESI+) (M+H)+=422

m.p. 309.10° C. (onset)

### Polymorphs Conversion (Hydrate to Form-4)

### 4-[4-(2-Adamantylcarbamoyl)-5-tert-butyl-pyrazol-1-yl]benzoic acid (form 4)

Tetrahydrofuran (10.0 rel. vol) was added to 4-[4-(2-adamantylcarbamoyl)-5-tert-butyl-pyrazol-1-yl] benzoic acid hydrate from the previous step (4.0 g, 0.009 mol) and then the reaction temperature raised to 58-62° C. Acetonitrile (20.0 rel. vol) was added whilst maintaining the reaction at 55-65° C. The reaction temperature was raised to 68±2° C., maintained there for 22 hours, then cooled to 20 to 25° C. and stirred for 2 hours. The product was filtered and the bed washed with acetonitrile (5.0 rel. vol). The wet cake was dried under vacuum (50-100 mbar) at 45 to 50° C. for 4 hours to give polymorph 4 (yield 80%) as confirmed by XRPD.

### Form 5

4. 20 g of 4-[4-(2-adamantylcarbamoyl)-5-tert-butyl-pyrazol-1-yl]benzoic acid was charged crude into a 500 ml reactor followed by tetrahydrofuran (180.00 mL) and water (10.00 ml). The reaction mass was stirred for 20 minutes and became a clear solution. The solution was filtered and a line wash off. tetrahydrofuran (20.00 mL) was added. The combined filtrate was charged into the reactor and the temperature of the reaction mass raised to 60° C.

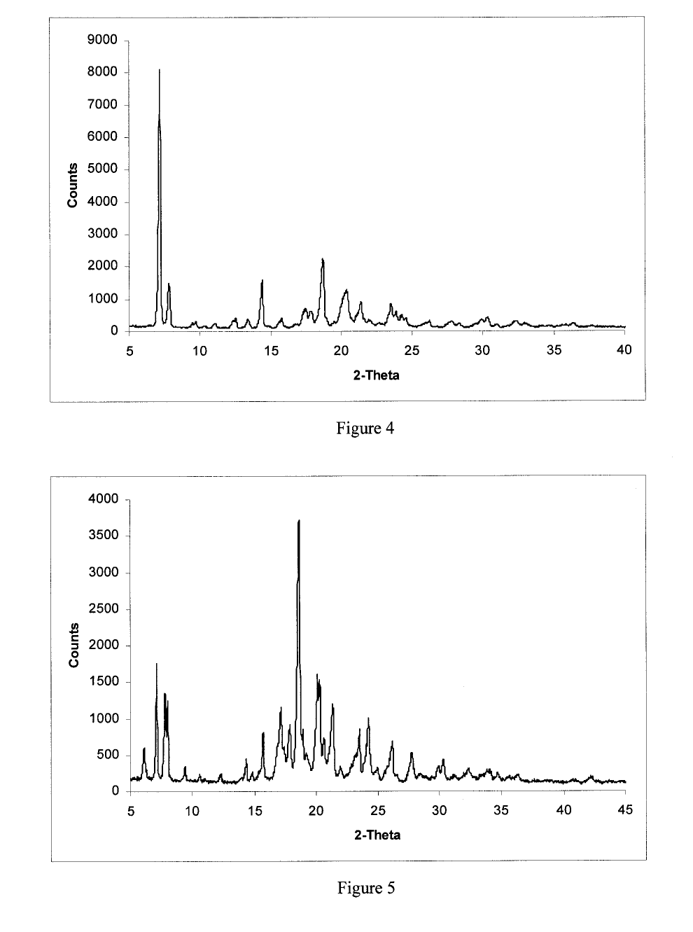

4-[4-(2-adamantylcarbamoyl)-5-tert-butyl-pyrazol-1-yl]benzoic acid, Form 4 seed was added to the solution and did not dissolve. The reaction mass was stirred for 1 hour until growth appeared to have stopped on the Lasentec probe. The reaction mass was cooled to 15° C. over 5 hours then stirred for one hour. The product (solid) was filtered under vacuum. The product was sucked dry for 5 minutes then dried under vacuum (50° C.) overnight. This solid was analyzed by XRPD (cobolt source) giving substantially the following d-values. See FIG. 4 for diffractogram.

### Peaks for Form 5

| d-spacing [Å] | Rel. Int. [%] |
|---|---|
| 14.3 | vs |
| 13.1 | m |
| 8.2 | vw |
| 7.7 | vw |
| 7.2 | m |
| 6.5 | vw |
| 5.9 | w |
| 5.8 | w |
| 5.5 | m |
| 5.0 | m |
| 4.82 | w |
| 4.40 | w |
| 4.25 | w |
| 4.20 | vw |

### Form 6

10 mL of tetrahydrofuran:water (10:0.5) was added to a polyblock vial containing ˜400 mg of 4-[4-(2-adamantylcarbamoyl)-5-tert-butyl-pyrazol-1-yl]benzoic acid of Form 4. The reaction mass was stirred at 22° C. for 2 days. All the solid had dissolved after two days. The solution was collected in a beaker. Within 5 minutes, solid crashed out of the solution. This solid was analyzed by XRPD (cobolt source) giving substantially the following d-values. See FIG. 5 for diffractogram.

### Peaks Form 6

| d-spacing [Å] | Rel. Int. [%] |
|---|---|
| 16.7 | m |
| 14.4 | s |
| 13.1 | s |
| 12.8 | m |
| 10.9 | vw |
| 7.2 | w |
| 6.9 | vw |
| 6.5 | m |
| 6.0 | m |
| 5.8 | m |
| 5.5 | vs |
| 5.13 | s |
| 5.08 | s |
| 5.00 | m |
| 4.82 | m |
| 4.69 | w |
| 4.39 | m |
| 4.26 | m |
| 3.95 | m |
| 3.73 | m |
| 3.42 | w |
| 3.21 | w |
| 3.05 | vw |

### Hemihydrate

### Polymorphs Conversion to Hemihydrate

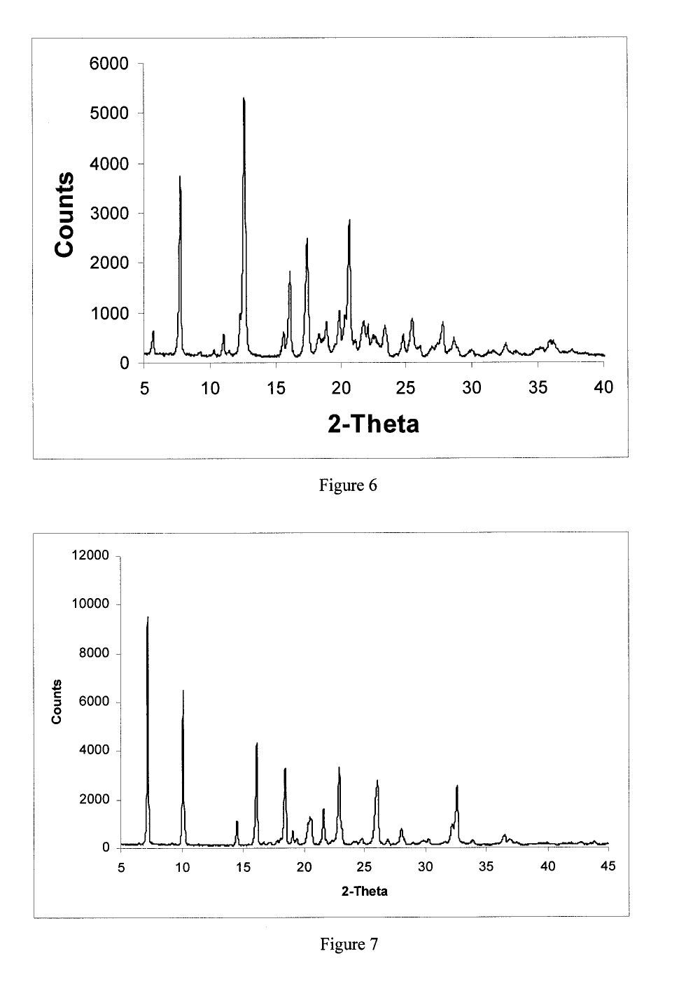

To the 4 necked RB flask equipped with stirrer and thermometer was charged 4-Hydrazino benzoic acid. HCl (2.88 g). Isopropyl Alcohol (50 mL) was charged into the reaction mass and the reaction mass was stirred for 5 minutes at 23-25° C. 4-[4-(2-adamantylcarbamoyl)-5-tert-butyl-pyrazol-1-yl]benzoic acid, enamine (5.1 g [Limiting Reagent]; 1.00 equivalents; 14.99 mmoles; 5.10 g; [Actual]) were dissolved in Isopropyl Alcohol (50 mL). Then the dissolved enamine solution was added to 4-hydrazino benzoic acid solution in IPA at 23-25° C. The reaction mass was stirred for 1 hour at 23-25° C. for 1 hour. The reaction mass was heated to reflux at 80° C. The reaction mass was maintained for 1 hour at a reflux temperature of 80° C. Once the reaction was over the reaction mass was cooled to 25° C. and stirred at 25° C. for 1 hour. 50 mL of purified water was added and stirred at 25° C. for 1 hour. Subsequently it was stirred for another 30 minutes. Thereafter it was filtered through a buckerner funnel. Isopropyl alcohol and water (50:50) (50 g) was given for washing. Then it was suck dried for 15 minutes. Thereafter it was dried under vacuum for 12 hours at 50° C. Moisture content was confirmed by KF. (2% w/w). The solid was analysed by XRPD (cobolt source) giving substantially the following d-values. See FIG. 6 for diffractogram.

### Peaks for Hemihydrate

| d-spacing [Å] | Rel. Int. [%] |
|---|---|
| 17.9 | w |
| 13.2 | s |
| 9.3 | w |
| 8.1 | vs |
| 6.6 | w |
| 6.4 | s |
| 5.9 | s |
| 5.6 | w |
| 5.4 | m |
| 5.2 | m |
| 4.98 | s |
| 4.74 | m |
| 4.67 | m |
| 4.58 | w |
| 4.42 | m |
| 4.17 | w |
| 4.06 | m |
| 3.72 | m |
| 3.62 | w |

### DMF Solvate

5.6 g of Form 1 of 4-[4-(2-adamantylcarbamoyl)-5-tert-butyl-pyrazol-1-yl]benzoic acid in 40 mL of dimethylformamide was heated it for two days at 50° C., then cooled at 25° C., filtered and dried in vacuum for 4 hours. TGA and DSC was performed on the sample, which indicated it to be a dimethylformamide-solvate. The sample was analysed by XRPD (Cobolt source) giving substantially the following d-values. For diffractogram see FIG. 7.

### Peaks for DMF Solvate

| d-spacing [Å] | Rel. Int. [%] |
|---|---|
| 14.2 | vs |
| 10.1 | s |
| 7.1 | m |
| 6.4 | s |
| 5.6 | s |
| 5.4 | w |
| 5.3 | vw |
| 5.1 | w |
| 4.99 | m |
| 4.77 | m |
| 4.50 | s |
| 4.46 | w |
| 4.17 | vw |
| 3.96 | m |
| 3.69 | w |
| 3.22 | w |
| 3.19 | m |

### EtOH-Solvate

Single crystal X-ray diffraction analysis, using Mo-source with wavelength 0.71073 Å and a graphite monochromator, showed EtOH-solvate to crystallize in the orthorhombic space group P2₁2₁2₁ with 4 molecules in the unit cell. The unit cell dimensions were found to be: a=7.0600(2)

b=16.1280(4)
c=22.0580(4)
α=90°
β13=90°
γ=90°

### V=2511.61 Å³

### Z=4

The calculated density is D_{c}=1.24 g/cm³.

### MeOH Solvate

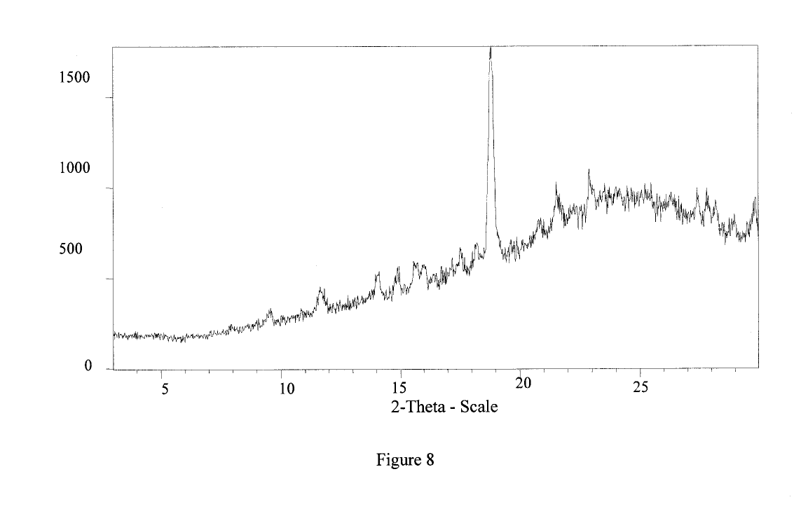

20 mg of Form 4 was added to a small vessel. 0.711 ml of Form 4 saturated MeOH solution was added to the vessel making a suspension. The suspension was stirred for 7 days at 25° C. Thereafter the suspension was stirred at 21° C. for 1½ months. The wet suspension was analysed by XRPD (Cu-source) giving substantially the following d-values. The material is instable and on exposure to the lab atmosphere (21° C., 30% relative humidity) this form rapidly transforms to Form 3. For diffractogram see FIG. 8.

### Peaks for Me OH-Solvate

| d-spacing (Å) | Relative intensity |
|---|---|
| 9.3 | w |
| 7.6 | m |
| 6.3 | m |
| 5.9 | m |
| 5.7 | m |
| 5.5 | w |
| 5.0 | m |
| 4.87 | m |
| 4.71 | vs |
| 4.27 | w |
| 4.1 | m |
| 3.88 | m |
| 3.25 | m |
| 3.21 | m |
| 3.16 | w |

### MTBE Solvate

20 mg of Form 4 was added to a small vessel. 0.711 ml MTBE was added to the vessel making a suspension. The suspension was stirred for 7 days at 25° C. The wet suspension was analysed by XRPD (Cu-source) giving substantially the following d-values. The material is instable and on exposure to the lab atmosphere (21° C., 30% relative humidity) this form transforms to Hydrate.

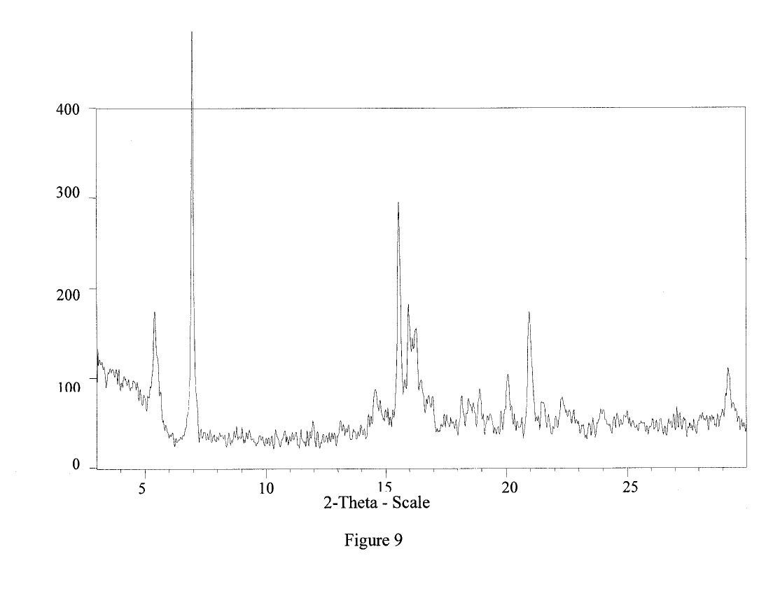

See FIG. 9 for diffractogram.

### Peaks for MTBE Solvate

| d- spacing (Å) | Relative intensity |
|---|---|
| 16.3 | m |
| 12.7 | vs |
| 5.7 | s |
| 5.5 | m |
| 5.4 | m |
| 4.42 | m |
| 4.23 | m |
| 3.05 | m |

### X-Ray Powder Diffraction

The X-ray diffraction (referred to herein as XRPD) analysis was performed according to standard methods, which can be found in e.g. Kitaigorodsky, A. I. (1973), Molecular Crystals and Molecules, Academic Press, New York; Bunn, C. W. (1948), Chemical Crystallography, Clarendon Press, London; or Klug, H. P. & Alexander, L. E. (1974), X-ray Diffraction Procedures, John Wiley & Sons, New York. X-ray powder diffraction data were measured without any internal reference.

The X-ray powder diffraction patterns was determined by mounting a thin layer of the sample on a zero background holder, single silicon crystal or on a stainless steel holder with 2 mm depth. The samples were spun (to improve counting statistics) and automatic variable divergence slits were used.

Form 5, Form 6, Hemi-hydrate and DMF-solvate were analysed using a PanAlytical X′Pert PRO theta-2 theta diffractometer with an Xcelerator detector. The X-rays were generated by a cobalt tube operated at 40 kV and 30 mA with a wavelength of 1.78901 angstroms.

MeOH-solvate and MTBE-solvate were analysed using a PanAlytical X′Pert PRO theta-2 theta diffractometer with an Xcelerator detector. The X-rays were generated by a copper tube operated at 40 kV and 30 mA with a wavelength of 1.5406 angstroms.

The X-ray powder diffraction (XRPD) patterns in this were obtained in Bragg-Brentano geometry. Persons skilled in the art of X-ray powder diffraction will realise that the relative intensity of peaks can be affected by, for example, grains above 30 microns in size and non-unitary aspect ratios that may affect analysis of samples. The skilled person will also realise that the position of reflections can be affected by the precise height at which the sample sits in the diffractometer and the zero calibration of the diffractometer. The surface planarity of the sample may also have a small effect.

It is known that an X-ray powder diffraction pattern may be obtained which has one or more measurement errors depending on measurement conditions (such as equipment or machine used). No internal standard was used in any of the XRPD analyses and therefore the diffraction pattern data presented are not to be taken as absolute values. In particular, it is generally known that intensities in an X-ray powder diffraction pattern may fluctuate depending on experimental conditions and sample preparation (e.g. preferred orientation). (Jenkins, R & Snyder, R. L. ‘Introduction to X-Ray Powder Diffractometry’ John Wiley & Sons 1996; Bunn, C. W. (1948), Chemical Crystallography, Clarendon Press, London; Klug, H. P. & Alexander, L. E. (1974), X-Ray Diffraction Procedures).

The following definitions of relative intensity have been used.

| % Relative Intensity | Definition |
|---|---|
| 81-100 | vs (very strong) |
| 31-80 | s (strong) |
| 11-30 | m (medium) |
| 6-10 | w (weak) |
| 3-5 | vw (very weak) |

1. A process for preparing a compound of formula (I) 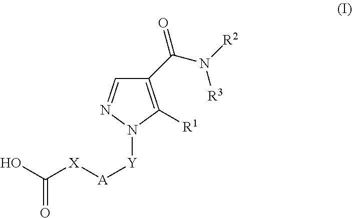
wherein:
R¹ is C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, C₃₋₇cycloalkylC₁₋₃alkyl, C₃₋₇cycloalkylC₂₋₃alkenyl or C₃₋₇cycloalkylC₂₋₃alkynyl, [each of which is optionally substituted by 1, 2 or 3 substituents independently selected from C₁₋₃alkyl, hydroxy, halo, oxo, cyano, trifluoromethyl, C₁₋₃alkoxy, C₁₋₃alkylS(O)ₙ— (wherein n is 0, 1, 2 or 3), R⁵CON(R^{5′})—, (R^{5′})(R^{5″})NC(O)—, R^{5′}C(O)O—, R^{5′}OC(O)—, (R^{5′})(R^{5″})NC(O)N(R^{5′″})—, R⁵SO₂N(R^{5″})—, and (R^{5′})(R^{5″})NSO₂— (wherein R⁵ is C₁₋₃alkyl optionally substituted by 1, 2 or 3 substituents selected from hydroxyl, halo or cyano; and
R^{5′} and R^{5″} are independently selected from hydrogen and C₁₋₃alkyl optionally substituted by 1, 2 or 3 substituents independently selected from hydroxyl, halo, C₁₋₃alkoxy, carboxy and cyano or R^{5′} and R^{5″} together with the nitrogen atom to which they are attached form a 4-7 membered saturated ring)];
R² is selected from heterocyclyl, C₃₋₇cycloalkyl(CH₂)ₘ—, and C₆₋₁₂polycycloalkyl(CH₂)ₘ— (wherein m is 0, 1 or 2 and the rings are optionally substituted by 1, 2 or 3 substituents independently selected from R⁶);
R³ is selected from hydrogen, C₁₋₄alkyl C₃₋₅cycloalkyl and C₃₋₅cycloalkylmethyl (each of which is optionally substituted by 1, 2 or 3 fluoro atoms);
R² and R³ together with the nitrogen atom to which they are attached form a saturated mono, bicyclic or bridged ring system optionally containing 1 or 2 additional ring heteroatoms selected from nitrogen, oxygen and sulphur and which is optionally fused to a saturated, partially saturated or unsaturated monocyclic ring wherein the resulting ring system is optionally substituted by 1, 2, or 3 substituents independently selected from R⁷;
R⁶ and R⁷ are independently selected from hydroxyl, halo, oxo, carboxy, cyano, trifluoromethyl, R⁹, R⁹O—, R⁹CO—, R⁹C(O)O—, R⁹CON(R^{9′})—, (R^{9′})(R^{9″})NC(O)—, (R^{9′})(R^{9″})N—, R⁹S(O)ₐ— wherein a is 0 to 2, R^{9′}OC(O)—, (R^{9′})(R^{9″})NSO₂—, R⁹SO₂N(R^{9″})—, (R^{9′})(R^{9″})NC(O)N(R^{9′″})—, phenyl and heteroaryl [wherein the phenyl and heteroaryl groups are optionally fused to a phenyl, heteroaryl or a saturated or partially-saturated 5- or 6-membered ring optionally containing 1, 2 or 3 heteroatoms independently selected from nitrogen, oxygen and sulphur and the resulting ring system is optionally substituted by 1, 2 or 3 substituents independently selected from C₁₋₄alkyl, hydroxyl, cyano, trifluoromethyl, trifluoromethoxy, halo, C₁₋₄alkoxy, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)ᵣ—, C₁₋₄alkylS(O)ᵣC₁₋₄alkyl (wherein r is 0, 1 or 2)];
R⁹ is independently selected from C₁₋₃alkyl optionally substituted by hydroxyl, halo, C₁₋₄alkoxy, carboxy or cyano;
R^{9′}, R^{9″} and R^{9′″} are independently selected from hydrogen and C₁₋₃alkyl optionally substituted by 1, 2, or 3 substituents independently selected from hydroxyl, halo, C₁₋₄alkoxy, carboxy and cyano);
A is a phenyl or heteroaryl ring (the phenyl or heteroaryl ring being optionally substituted on ring carbon atoms by 1, 2 or 3 R¹⁰ groups and on an available ring nitrogen in a heteroaryl group by R¹¹);
R¹⁰ is independently selected from C₁₋₄alkyl, hydroxyl, cyano, trifluoromethyl, trifluoromethoxy, halo, C₁₋₄alkoxy, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)ₛ—, C₁₋₄alkylS(O)ₛC₁₋₄alkyl (wherein s is 0, 1 or 2)];
R¹¹ is independently C₁₋₃alkyl optionally substituted by 1, 2 or 3 fluoro atoms;
X is a direct bond, C₃₋₄cycloalkandiyl, C₃₋₄cycloalkanylidene, —C(R¹²)(R¹³)—,
—C(R¹²)(R¹³)C(R¹⁴)(R¹⁵)—, —CH₂O— or —CH₂S(O)ₜ— (wherein t is 0, 1 or 2):
Y is a direct bond, C₃₋₄cycloalkandiyl, C₃₋₄cycloalkanylidene, —C(R¹⁶)(R¹⁷)— or —C(R¹⁸)(R¹⁹)C(R²⁰)(R²¹)—;
wherein R¹², R¹³, R¹⁴, R¹⁵, R¹⁶, R¹⁷, R¹⁸, R¹⁹, R²⁰ and R²¹ are independently selected from hydrogen and methyl; which process comprises reacting a compound of formula (II) 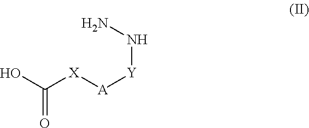
where X and A are as defined in relation to formula (1), with a compound of formula (III) 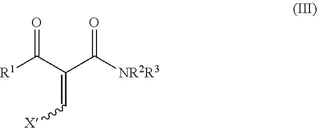
wherein R¹, R² and R³ are as defined above, and X′ represents either dialkylamino (such as dimethylamino) or lower alkoxy (such as methoxy or ethoxy);
and thereafter if necessary or desirable carrying out one or more of the following steps:
i) converting a compound of the formula (1) into another compound of the formula (1);
ii) removing any protecting groups;
iii) resolving enantiomers;
iv) forming a pharmaceutically-acceptable salt thereof; and
v) purifying the product.

2. A process according to claim 1 wherein the compound of formula (III) is obtained by reacting a compound of formula (IV) 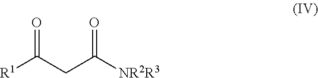
where R¹, R² and R³ are as defined above, with an acetal of formula (V) 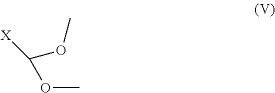
where X′ is as defined above.

3. A process according to claim 2 wherein the reaction is carried out in toluene or a mixture of toluene and n-heptane, and the product isolated by addition of a anti-solvent.

4. A process according to claim 3 wherein the anti-solvent is heptane.

5. A process according to claim 1 wherein the compound of formula (I) is a compound of formula (IA): 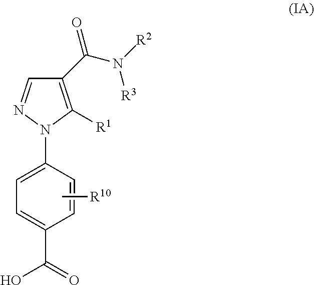
wherein R¹, R² and R³ are as defined in claim 1, and R¹⁰ is selected from hydrogen, C₁₋₄alkyl, trifluoromethyl, C₁₋₄alkoxy and C₁₋₄alkylS-.

6. A process according to claim 1 wherein R¹ is tert-butyl.

7. A process according to claim 1 wherein R² is adamantyl.

8. A process according to claim 1 wherein R³ is hydrogen.

9. A process according to claim 1 wherein Y is a direct bond.

10. A process according to claim 1 wherein A is phenyl optionally substituted by R¹⁰.

11. A process according to claim 1 wherein X is a direct bond.

12. A process according to claim 1 wherein the compound of the formula (I) is 4-[4-(2-adamantylcarbamoyl)-5-tert-butyl-pyrazol-1-yl] benzoic acid or a pharmaceutically-acceptable salt thereof, which process comprises the step of reacting a compound of the formula (IIB): 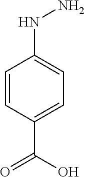
or salt thereof;
with a compound of formula (IIIB): 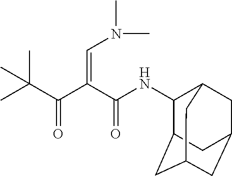
and thereafter if necessary or desirable carrying out one or more of the following steps:
i) forming a pharmaceutically-acceptable salt thereof; and
ii) purifying the product.

13. A process according to claim 12 wherein no intermediate is isolated.

14. A process according to claim 1 wherein the product is purified by aqueous workup comprising acidification of aqueous NaOH solution containing product.

15. A process according to claim 1 wherein the product is obtained is in polymorphic form by heating a suspension of purified product in acetonitrile or acetone.

16. A process for preparing a compound of formula (IV) as defined in claim 2, which process comprises reacting a compound of formula (VI) 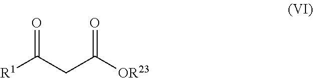
where R¹ is as defined in claim 1 and R²³ is an alkyl group; with a compound of formula (VII) 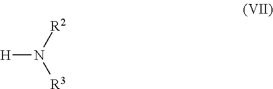
wherein R² and R³ are as defined in claim 1.

17. A process according to claim 15 which is effected in toluene and the compound of formula (VI) is isolated by addition of an anti-solvent.

18. A process according to claim 16 wherein the anti-solvent is n-heptane.

19. A process according to claim 15 wherein the compound of formula (VII) is generated in situ by addition of a base to a solution of a salt, for example an acid addition salt such as a hydrochloride salt of a compound of formula (VII).
